(19) United States
(12) Patent Application Publication
(10) Pub. No.: US 2016/0053216 A1
(43) Pub. Date: Feb. 25, 2016

(54) A MACHINE ENABLING SEPARATION OF THE BLOOD BY MAGNETISM AND AUTONOMOUS RADIOACTIVE SUBSTANCE MARKING OF THE WHITE BLOOD CELLS

(71) Applicant: SANKO OZEL EGITIM HIZMETLERI A.S., Gaziantep (TR)

(72) Inventors: Orhan ÇEVIK, Gaziantep (TR) (TR); Melike DEMIRCI, Gaziantep (TR) (TR)

(21) Appl. No.: 14/779,455

(22) PCT Filed: Mar. 27, 2014
(86) PCT No.: PCT/TR2014/000090

§ 371 Date: Sep. 23, 2015

(30) Foreign Application Priority Data
Apr. 3, 2013 (TR) 2013/04037

(51) Int. Cl. C12M 1/26

(52) U.S. Cl. CPC C12M 33/10

(57) ABSTRACT

A machine having a housing and an inner receptacle provided inside the housing with at least one tube therein holding the blood desired to be separated for separation of blood into white blood cells. The machine includes the following: at least one coil provided with at least one tube slot formed thereon so as to house the at least one tube and creating a magnetic field on the tube by means of the electric current passed therethrough; at least one motor enabling the coil to be rotated around its own axis so as to create a centrifugal effect on the tube; and at least one injector enabling the separated blood components to be transferred into the determined tubes by entering into the tubes.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to the machines, in particular, to the machines of the prior art part of claim 1 facilitating the separation of blood into white blood cells.

2. Description of Related Art Including Information Disclosed Under 37 CFR 1.97 and 37 CFR 1.98.

Blood is actually a tissue circulating in a closed system of channels in the organism. Each body is provided with 5-6 liters of blood. This amount constitutes 7-8% of the average body weight. A large part of the blood (55-60%) comprises the liquid portion, i.e. the plasma. The other half thereof comprises cells or molecules serving for various tasks inside the blood such as transportation, regulation and protection.

Blood plasma is composed of 91% water, 8% organic material and 1% inorganic material. Almost all of the organic compounds are proteins and plasma is formed by the proteins dissolved in water. The blood cells are composed of cells such as erythrocytes, leukocytes and thrombocytes. Erythrocytes (red blood cells) have a disk-shaped structure without nucleus. Leukocytes (white blood cells) are composed of mononuclear cells and polymorphonuclear cells. Thrombocytes (blood platelets) are cell fragments derived from megakaryocytes. They play a role in the clot formation.

Centrifugation process is carried out in laboratory settings for separating the blood into the aforementioned components thereof. Centrifugation is realized by means of an apparatus performing a fixed axis circular motion usually with the aid of an electric motor. The centrifugal force created by the rotational motion performed by the centrifugation apparatus enables separation of particles inside the blood with different specific gravities. However, centrifugation processes performed as such take a very long period of time. This is because same processes are sequentially performed on each tube in the current centrifuge machines. In other words, simultaneous processing of the tubes is not possible. This situation prolongs the process.

In the patent application TR 2011/08339, a method for separation of white blood cells from the blood is disclosed. Said invention relates to a method for reducing the number of white blood cells by means of processing the blood in an organic polymer based multifilament arrangement. According to said method, the blood flows towards the filaments and the number of white blood cells in blood is greatly reduced as a result of the absorption thereof by the filaments. Said invention is characterized in that the method with the inventive step comprises provision of organic polymer based multifilaments inside a receptacle comprising an input device and an output device, wherein at this stage, when blood is taken into the receptacle through the input device, it flows through the receptacle provided with organic polymer based multifilaments therein and exits through the output device on the other side. In addition, arrangement of the filaments is carried out according to a very notable layout, wherein blood is guided over the outer surface of the filaments and thus the complementary activation product C5a of the filaments is provided with a concentration of at least in laboratory settings 10 pg per 5 m2 of the filament surface.

In conclusion, due to the aforementioned adverse situations, a need to provide a machine enabling easy separation of white blood cells from the blood and thus easy marking of the white blood cells has emerged.

### BRIEF SUMMARY OF THE INVENTION

The present invention relates to a machine enabling separation of blood into white blood cells, eliminating all the aforementioned drawbacks as well as providing some additional benefits to the respective technical field.

The main object of the present invention is to provide a machine performing separation of blood into white blood cells as well as marking of the white blood cells in a shorter period of time.

The present invention, in order to achieve all the objects, which are mentioned above and will be evident from the following detailed description, relates to a machine comprising a housing and an inner receptacle provided inside said housing with at least one tube therein holding the blood desired to be separated for separation of blood into white blood cells. Said machine is provided with the following: at least one coil provided with at least one tube slot formed thereon so as to house at least one said tube and creating a magnetic field on the tube by means of the electric current passed there through, at least one motor enabling said coil to be rotated around its own axis so as to create a centrifugal effect on the tube and at least one injector enabling the separated blood components to be transferred into the determined tubes by entering into the tubes.

The present invention, in a preferred embodiment thereof, comprises a display panel showing various parametric values. The user is provided with required information such as amount of blood, amount of leukocytes, etc. by means of said display panel.

The present invention, in another preferred embodiment thereof, comprises a control button initiating the electric current supplied to the coil and actuating the motor enabling the coil to rotate. Furthermore, the present invention comprises a third control button enabling the plasma portion of the blood to be transferred into a separate tube and the white blood cells of the blood to be transferred into another tube and a power button enabling the machine to be turned on and off.

The present invention also relates to a method enabling separation of blood into white blood cells, wherein it comprises the following process steps:
a) Filling the blood into a tube and applying a magnetic field on the blood
b) Rotating the tube and/or blood so as to create a centrifugal effect
c) Transferring the plasma portion of the blood into a separate tube by inserting an injector into the tube
d) Transferring the white blood cells of blood into another tube provided with tec-99 therein by inserting an injector into the tube

### BRIEF DESCRIPTION OF THE DRAWINGS

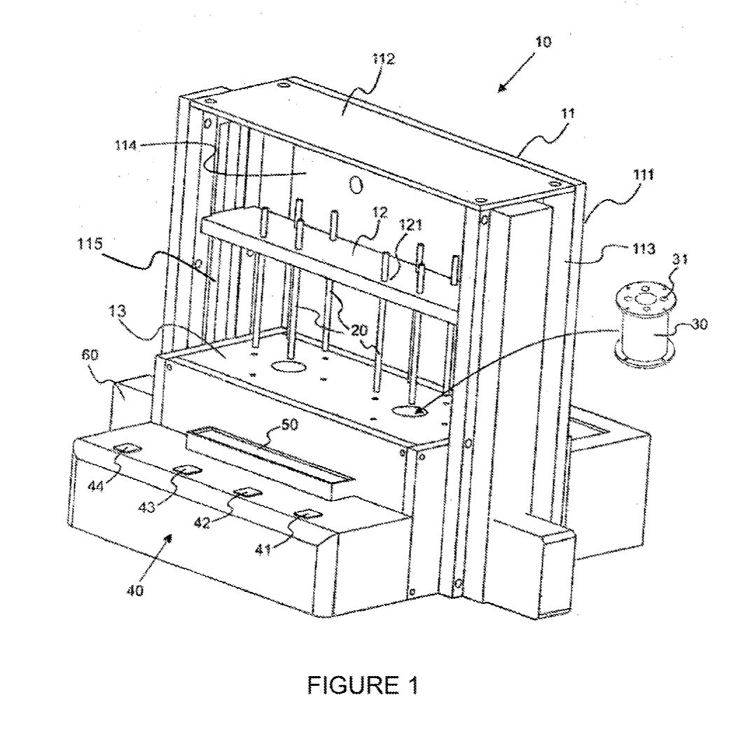

In FIG. 1, a general view of the machine according to the present invention is given.

### REFERENCE NUMBERS

10 Machine
11 Housing
111 Rear cover
112 Upper cover
113 Side cover
114 Inner receptacle
115 Guideway
12 Upper plate
121 Hole
13 Lower plate
20 Injector
30 Coil
31 Tube slot
40 Control buttons
41 First control button
42 Second control button
43 Third control button
44 Power button
50 Display panel
60 Motor

### DETAILED DESCRIPTION OF THE INVENTION

In this detailed description, the machine (10) according to the present invention is described only for a better understanding of the subject without constituting any restrictive effect. Accordingly, the machine (10) is assumed to be implemented so as to facilitate the separation of blood into the white blood cells. However, the present invention can also be implemented to facilitate separation of different substances in the same manner with minor revisions.

In FIG. 1, general view of the machine (10) is provided. Accordingly, in most general sense, the machine (10) comprises a housing (11) comprising a rear cover (11), two side covers extending in continuation of the rear cover (11) and an upper cover (112) provided above these such that a front face thereof remains open. Said upper cover and two side covers (111, 112, 113) define an inner receptacle (114).

A coil (30) enabling application of magnetic field to the blood is provided. Wire is wound around the coil (30). Thus, magnetic field can be applied on the blood. A tube slot (31) is provided on the coil (30) so as to enable the injectors (20) to be attached to the coil (30). In the embodiment according to the present invention, two coils (30) are placed side by side into the inner receptacle (114) of the machine housing (11). Preferably, four tube slots (31) are provided in each coil (30). However, in the alternative embodiments of the present invention, more or less number of tube slots (31) can be formed on the coil (30). The tubes (not shown in the figure) are placed into said tube slots (31). Blood desired to be separated is placed into the tubes.

The injectors (20) comprise single use needles. Vacuum hoses (not shown in the figure) are then attached on said needles. The blood inside the tube slots (31) are taken into the plastic pipes by means of the injectors (20). Plasma of the blood is transferred into a tube provided at the rear portion and white blood cells thereof is transferred into another tube comprising tec-99 therein for marking the white blood cells by means of the sensitive sensors connected to said pipes. Erythrocytes and thrombocytes of the blood are left in the first tube.

The injectors (20) are disposed inside the inner receptacle (114) of the housing (11) on an upper plate (12). The upper plate (12) comprises holes (121) for placement of the injectors (20) thereon. The number of holes (121) is equal to the number of injectors (20) desired to be placed. The upper plate (12) is movable up and down in the vertical direction. Movement of the upper plate (12) in the vertical direction is provided by the guideways (115) defined on the side covers (113) of the housing (11). Said guideways (115) are in the form of grooves formed on the side covers (113). A protrusion form entering into said groove is provided on the upper plate (12). A lower plate (13) is disposed at the lower portion of the upper plate (12). Coils (30) are placed on the lower plate (13). The lower plate (13) and upper plate (12) are positioned such that the holes (131) and tube slots (31) are mutually aligned with each other. Thus, after the movement of the upper plate (12), the injectors (20) can enter into the tube slots (31).

Coils (30) are rotated around their own axis in order to create a centrifugal effect on the blood. Thus, separation of the blood is accelerated by means of the magnetic field and centrifugal effect. A motor (60) is provided for rotating the coil (30). The motor (60) preferably operates with electricity. However, in the alternative embodiments of the present invention a motor (60) operated with different power sources can also be used.

Control buttons (40) actuating the machine (10) are provided. Among said control buttons (40), the first control button (41) initiates the electric current supplied to the first coil (30). Further, it actuates the motors (60) connected to the first coil (30) and initiates rotation of the first coil (30) around its own axis. Thus, separation in the first coil (30) is realized. The second control button (42), as the first control button (41), also initiates the electric current supplied to the second coil (30). Further, it actuates the motors (60) connected to the second coil (30) and initiates rotation of the second coil (30) around its own axis.

A third control button (43) enabling the plasma portion of the blood to be transferred into a separate tube disposed at the rear portion of the housing (11) and the white blood cells of the blood to be transferred into another tube disposed at the rear portion of the housing (11) and comprising tec-99 therein is provided. White blood cells are marked by means of the third control button (43). A power button (44) enabling the machine (10) to be turned on and off is provided. Said button turns the machine (10) on and off.

A display panel (50) enabling the information to be visually provided to the user is disposed so as to be connected to the machine housing (11). Informative messages including amount of blood, amount of white blood cells and/or facilitating utilization of the machine (10) can be displayed to the user through the display panel (50).

In view of the above description, implementation of the present invention is carried out as follows. First, bloods desired to be separated are placed into the tubes. Then, the tubes are placed into the tube slots (31) disposed on the coil (30). Following, electricity is supplied to the coil (30), thus, generation of a magnetic field is provided by means of the wire wound around the coil (30). Then, coil (30) is rotated around its own axis by means of the motor (60). With said rotation, centrifugal effect is provided to the blood simultaneously with the magnetic field. After these processes are completed, the injectors (20) enter into the tubes, by means of the sensors, plasma of the blood is transferred into a tube provided at the rear portion of the housing (11) and white blood cells thereof is transferred into another tube comprising tec-99 therein for marking the white blood cells. Erythrocytes and thrombocytes of the blood are left in the first tube. Thus the process is completed.

In conclusion, thanks to the present invention, separation of blood into the white blood cells will be facilitated and thus the processes will be carried out in a more healthy manner as well as time will be saved.

1. A machine comprising a housing and an inner receptacle provided inside said housing with at least one tube therein holding the blood desired to be separated for separation of blood into white blood cells, characterized in comprising:
at least one coil provided with at least one tube slot formed thereon so as to house at least one said tube and creating a magnetic field on the tube by means of the electric current passed therethrough;
at least one motor enabling said coil to be rotated around its own axis so as to create a centrifugal effect on the tube; and
at least one injector enabling the separated blood components to be transferred into the determined tubes by entering into the tubes.

2. The machine according to claim 1, characterized in that said tube slot can be configured such that a plurality of tube slots with different sizes from one another is provided.

3. The machine according to claim 1, characterized in that it comprises an upper plate positioned inside the inner receptacle and provided with at least one injector thereon.

4. (canceled)

5. The machine according to claim 4, characterized in that said holes are configured in large numbers so as to be able to utilize more than one injector.

6. The machine according to claim 2, characterized in that it comprises guideways defined on side covers for enabling the upper plate to move up and down in the vertical direction along the side covers of the housing.

7. The machine according to claim 1, characterized in that it comprises a lower plate allowing placement of said coil inside an inner receptacle so as to be provided with a freedom of motion to perform a rotational motion around its own axis.

8. The machine according to claim 5, characterized in that said upper plate and lower plate are positioned such that the holes and tube slots are mutually aligned with each other.

9. The machine according to claim 1, characterized in that it comprises at least one control button initiating the electric current supplied to the coil and actuating the motor enabling rotation of the coil.

10. The machine according to claim 1, characterized in that the number of said coils is two.

11. The machine according to claim 10, characterized in that it comprises a first control button enabling the control of the first coil and a second control button enabling the control of the second coil.

12. The machine according to claim 1, characterized in that it comprises a third control button enabling the plasma portion of the blood to be transferred into a separate tube disposed at the rear portion of the housing (and the white blood cells of the blood to be transferred into another tube.

13. The machine according to claim 1, characterized in that it comprises a power button enabling the machine to be turned on and off.

14. The machine according to claim 1, characterized in that it comprises a display panel showing various parametric values regarding the machine.

15. The machine according to claim 1, characterized in that it comprises a tube being filled with white blood cells of the blood and comprising tec-99 therein to enable marking thereof.

16. A method enabling separation of blood into white blood cells, characterized in that it comprises the following process steps:
a) Filling the blood into a tube and applying a magnetic field on the tube;
b) Rotating the tube simultaneously with the magnetic field application so as to create a centrifugal effect on the blood;
c) Transferring the plasma portion of the blood into a separate tube by inserting an injector into the tube; and
d) Transferring the white blood cells of blood into another tube provided with tec-99 therein by inserting an injector into the tub,
